(19) United States
(12) Patent Application Publication
(10) Pub. No.: US 2006/0204438 A1
(43) Pub. Date: Sep. 14, 2006

(54) Water soluble iron oxide nanoparticles and a method of its preparation

(75) Inventors: Sun Hang Cho, Daejeon (KR); Hae Bang Lee, Daejeon (KR); Gil Son Khang, Daejeon (KR); Moon Suk Kim, Daejeon (KR); Ha Young Lee, Jeonju-si (KR); Nak Hyun Lim, Iksan-si (KR); Jin A. Seo, Daejeon (KR); Seong Mi Seo, Miryang-si (KR)

Correspondence Address: FROMMER LAWRENCE & HAUG, 745 FIFTH AVENUE- 10TH FL., NEW YORK, NY, 10151, US

(73) Assignee: Korea Research Institute of Chemical Technology

(21) Appl. No.: 11/178,766

(22) Filed: Jul. 11, 2005

(30) Foreign Application Priority Data
Mar. 14, 2005 (KR) 10-2005-0021150

(51) Int. Cl. A61K 49/00

(52) U.S. Cl. 424/9.1; 424/9.34; 977/930

(57) ABSTRACT

This invention relates to water soluble iron oxide nanoparticles and a method of its preparation. More specifically, this invention relates to a method of manufacturing water soluble iron oxide nanoparticles uniform in size by coating polyvinylpyrolidone, a biocompatible polymer that can be intravenously administered via thermal decomposition, on the surface of iron oxide thereby enabling to control the size of resulting particles, and water soluble iron oxide nanoparticles coated with polyvinylpyrolidone. The above-mentioned water soluble iron oxide nanoparticles have excellent image contrasting effect and are thus useful as MRI contrast agent.

### CROSS REFERENCE TO RELATED APPLICATIONS

This application claims priority of Korean Application No. 10-2005-021150, filed Mar. 14, 2005, the disclosure of which is incorporated herein by reference.

### TECHNICAL FIELD OF THE INVENTION

This invention relates to water soluble iron oxide nanoparticles and a method of its preparation. More specifically, this invention relates to a method of manufacturing water soluble iron oxide nanoparticles which are uniform in size by coating polyvinylpyrolidone, a biocompatible polymer that can be administered intravenously via thermal decomposition, on the surface of iron oxide thereby capable of controlling the size of resulting particles, and water soluble iron oxide nanoparticles coated with polyvinylpyrolidone.

### BACKGROUND OF THE INVENTION

Generally, iron oxide nanoparticles are required to have excellent water solubility and a relatively narrow size distribution for their use in medical industry. For example, the nanoparticles should have a particle size of about 50 to 200 nm to be used as contrast agent for liver, and the particles are promptly removed from the blood due to phagocytosis by reticuloendothelial cells within an hour, and their distribution is liver-specific and thus can be used as contrast agent for liver. For iron oxide particles with a size of less than 50 nm, the above-mentioned phagocytosis by the phagocytes is relatively low and thus they can remain in blood for a relatively long period of time. Therefore, due to the rather lengthy retention in the blood they can provide a good image contrast of lymph nodes as well as that of blood. MRI contrast agent is used to increase image contrast between a normal tissue and a damaged tissue. In general, particles comprising para-gadolinium, manganese ions, colloidal magnetic nanoparticles, and superparamagnetic particles are intravenously injected to patients. Of magnetic nanoparticles, iron oxide particles in particular have excellent image contrast effect and thus have been used as MRI contrast agent. Besides, they can be used in targeted drug delivery, diagnosis and therapeutic treatment of cancer, thermotherapy, tissue and cell therapy, and the like.

The polyvinylpyrolidone of the present invention, having its chemical name of poly(1-vinyl-2-pyrolidone), is a polymer having properties similar to those of PEG and is a non-toxic and non-ionic biocompatible polymer with an excellent water solubility. The scope of its applications includes pharmaceutical excipients, food additives, stabilizers, and injectional preparations and thus it can be used as a stabilizer in the event of manufacturing MRI contrast agent.

Superparamagnetic contrast agent can be manufactured by various methods such as coprecipitation of iron salts and microemulsion in coating polymer solution such as dextran, and laser-induced thermal decomposition. However, thus obtained nanoparticles are not desirable because they are not uniform in size and also have relatively low crystallinity thus negative impact on the magnetic properties. Therefore, there were developed recently methods of thermal decomposition of iron oxide precursors such as FeCup₃, Fe(acac)₃ and Fe(CO)₅. However, iron oxides synthesized by the above methods are well dissolved in organic solvents and thus there are limitations in their applications to biomedical uses.

### SUMMARY OF THE INVENTION

The inventors of this invention have performed extensive researches to develop iron oxide nanoparticles which can resolve the above limitations in their applications to medical fields because they are water insoluble but soluble only in organic solvents. As a result, they have succeeded in manufacturing iron oxide nanoparticles which can control the particle size based on the established factors such as molecular weight of polypyrolidone, selection of an appropriate solvent and the content of precursor, are water soluble and also uniform in size, by coating polypyrolidone, a biocompatible polymer that can be intravenously injected, on the surface of iron oxide nanoparticles via thermal decomposition. Therefore, an object of the present invention is to provide a method for manufacturing iron oxide nanoparticles coated with polypyrolidone.

### BRIEF DESCRIPTION OF THE DRAWINGS

The above and other objects and features of the present invention will become apparent from the following description of the invention, when taken in conjunction with the accompanying drawings, wherein:

FIG. 1a shows a picture of iron oxide nanoparticles coated with polyvinylpyrolidone prepared in Example 1 taken by Transmission Electron Microscope;

FIG. 1b shows a picture of iron oxide nanoparticles coated with polyvinylpyrolidone prepared in Example 2 taken by Transmission Electron Microscope;

FIG. 2 shows an image contrast picture of a rabbit liver taken via MRI before and after addition of iron oxide nanoparticles coated with polyvinylpyrolidone prepared in Example 2; and

FIG. 3 shows that uniform particles cannot be obtained in Comparative Example 1 in manufacturing iron oxide due to the aggregation.

### DETAILED DESCRIPTION OF PREFERRED EMBODIMENTS

This invention relates to iron oxide nanoparticles coated with polypyrolidone which are water soluble and uniform in size and a method of their preparation.

The present invention is explained in greater detail as set forth hereunder.

In a preferred embodiment, the present invention provides water soluble iron oxide nanoparticles uniform in size obtained by coating polyvinylpyrolidone, a biocompatible polymer that can be intravenously administered on the surface of iron oxide via thermal decomposition thereby enabling to control the size of resulting particles, and water soluble iron oxide nanoparticles coated with polyvinylpyrolidone.

In manufacturing iron oxide nanoparticles, if they are to be applied to medical fields such as contrast agent, they should have a uniform distribution of particle size and thus it is very important that the size of iron oxide nanoparticles be controlled for that purpose. As a way to control the size of iron oxide nanoparticles the inventors of the present invention optimized such factors as the content of precursor, selection of a solvent, molecular weight of a stabilizer and the like.

In particular, polyvinylpyrolidone, the polymer to be used as a stabilizer in this invention, is adsorbed to the surface of growing metal particles and becomes stabilized thereby preventing growth and aggregation of metal particles and also determining the strength of interfacial interaction between polymer layers to be adsorbed to metal cluster by the flexibility of polymer chain. Besides, the viscosity of a solution can determine the overall reaction rate and thus the forms and shapes of the iron oxide nanoparticles to be obtained were shown to vary greatly depending on the molecular weight of polyvinylpyrolidone and a solvent.

In the present invention, there is provided a method for manufacturing water soluble iron oxide nanoparticles comprising: (a) heating a mixture consisting of polyvinylpyrolidone and its solvent at about 120 to 600° C., (b) adding an iron oxide precursor to the above mixture and agitating for about 30 min to 48 hrs, (c) cooling down to about 20 to 30° C. to obtain iron oxide nanoparticles coated with polyvinylpyrolidone on the surface.

The polyvinylpyrolidone used in the present invention is a polymer, similar to PEG in properties, which is a biocompatible, water soluble and non-toxic and non-ionic polymer. Further, its scope of applications include pharmaceutical excipients food additives, stabilizers, injectional preparations and thus it can be used as a stabilizer in manufacturing MRI image contrast medium.

The polyvinylpyrolidone is preferred to have molecular weight of 3,000-100,000 g/mol, more preferably 5,000-30,000 g/mol. This is because the longer the length of the polymer chain the stronger the adsorption with a relatively thin layer. If the molecular weight of polyvinylpyrolidone is less than 3,000 g/mol its length becomes insufficient as a chain for coating iron oxide thus causing aggregation and not being able to form particles uniform in size. Meanwhile, if the molecular weight is higher than 100,000 g/mol it results in aggregation among polymers thus not desirable.

Any polar organic solvent may be used as the above solvent, however, it is preferred that the above solvent to be selected from the group consisting of carbitol, glycol, dimethylformamide, propylenecarbonate, isopropylalcohol and glycerol. Examples of the above glycol include polyethyleneglycol, ethyleneglycol, and propylenecarbonate.

For the complete dissolution of the polyvinylpyrolidone, the mixture of polyvinylpyrolidone and its solvent is preferably heated at about 120 to 600° C., more preferably at about 150 to 300° C. If it is heated below 120° C. the resulting iron oxide precursor is not completely decomposed while it becomes carbonized if it is heated above 600° C.

Examples of an iron oxide precursor to be used in the present invention are FeCup₃, Fe(acac)₃, and Fe(CO)₅.

The iron oxide precursor is added to the above mixture consisting of polyvinylpyrolidone and its solvent and agitated for about 0.5 to 72 hrs to allow a reaction. Here, if the reaction time is less than 0.5 hour it results in incomplete formation of iron oxide while it results in production of relatively big sized-particles if the reaction time exceeds 72 hrs.

The rate of agitation is preferred to be performed in the range of about 300-500 rpm. If the agitation rate is lower than 300 rpm reactants are not able to make even contact with one another and thus sufficient coating cannot be achieved. If the agitation is performed in a relatively high speed it is highly likely that aggregation would occur due to insufficient coating.

In a preferred embodiment of the present invention, water soluble iron oxide nanoparticles are obtained by reacting polyvinylpyrolidone and iron oxide precursor in a molar ratio of about 1:0.01 to 1:10. If the molar ratio is below the above ratio it results in prevention of forming iron oxide core. Meanwhile, if the molar ratio is greater than the above ratio the content of a stabilizer becomes insufficient, which then causes the resulting iron oxide nanoparticles ununiform and irregular in shape.

In another preferred embodiment of the present invention, there are provided water soluble iron oxide nanoparticles coated with polyvinylpyrolidone, a biocompatible polymer, which can be also controlled in their size upon production. The limiting factors used in controlling the size of the iron oxide nanoparticles are the molar ratio between polyvuinylpyrolidone and iron oxide precursor, molecular weight of the polyvinylpyrolidone, reaction temperature, reaction time and the like. The size of the iron oxide nanoparticles can be controlled in the range of 1 to 500 nm, they can be produced uniformly in size and are water soluble, and all the above reactions are performed under nitrogen atmosphere.

Thus obtained water soluble iron oxide nanoparticles of 1 to 500 nm in size have excellent biomedical applications due to their superparamagnetism.

As shown in the following Examples, it was confirmed that the iron oxide nanoparticles coated with polyvinylpyrolidone have excellent image contrast effect around liver area from an experiment where they were injected to leg veins of a rabbit and therefore they can be used as an MRI contrast agent. Further, they can be also used in targeted drug delivery, therapeutic treatments of cells and tissues, thermotherapy and the like.

### EXAMPLE

This invention is explained in more detail based on the following Examples however they should not be construed as limiting the scope of this invention.

### Example 1

One gram of polyvinylpyrolidone Kollidone 17PF (K 15.3-18.0, MW; 7000-11000 g/mol, BASF, Germany) as a stabilizer was dissolved in 3 mL of dimethylformamide, added into a 100 mL three-necked round flask and refluxed. After heating the flask to 160° C., 0.052 mL of iron pentacarbonyl was added thereto using a syringe and stirred at 160° C. for about 2 hrs at 350 rpm, wherein the molar ratio between polyvinylpyrolidone and iron pentacarbonyl was 1:4. As the reaction started the initially orange-colored reactants were gradually changed to a dark brown colloidal solution of iron oxide. Upon completion of the reaction it was cooled down to room temperature to terminate the reaction and all the procedure was performed under the ultrapure nitrogen atmosphere. After the reaction, products were inserted into a dialysis membrane, Spectra/Por Membrane (Spectrum Laboratories, Inc., USA) with MWCO(molecular weight cut-off) 50000 and dialyzed by changing water every 3 hrs in the tertiary distilled water for a period of 24 hrs to remove unreacted polymers and solvents. The resulting dialyzed solution was frozen to −80° C. by using a deep freezer and then dried using a vacuum freeze dryer thereby removing the remaining solvents completely and obtaining powered product as a result. Thus obtained iron oxide power coated with polyvinylpyrolidone was well dissolved in water (powder A).

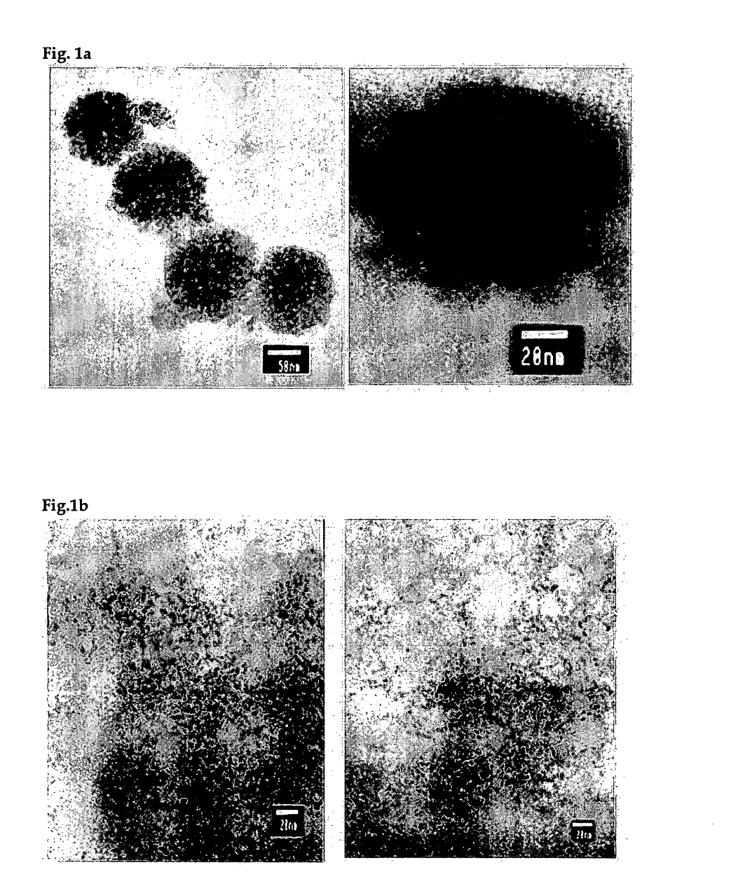

The picture of the powder A taken by Transmission Electron Microscope is shown in FIG. 1a and it was observed that uniform and spherical cores of iron oxide nanoparticles of 50 to 100 nm in size were formed. The x-ray diffraction of the above powder showed that there were mixtures of iron oxides with both divalent and trivalent irons.

Dynamic light scattering (DLS) analysis of the powder A in colloidal state revealed that the average size of iron oxide nannoparticles coated with polyvinylpyrolidone was 230 nm.

### Example 2

One gram of polyvinylpyrolidone Kollidone 17PF (K 15.3-18.0, MW; 7000-11000 g/mol, BASF, Germany) as a stabilizer was dissolved in 3 mL of carbitol (TCI, Tokyo, Japan), added into a 100 mL three-necked round flask and refluxed. After heating the flask to 160° C., 0.104 mL of iron pentacarbonyl was added thereto using a syringe and stirred at 160° C. for about 2 hrs at 350 rpm, wherein the molar ratio between polyvinylpyrolidone and iron pentacarbonyl was 1:8. Upon completion of the reaction it was cooled down to room temperature to terminate the reaction and all the procedure was performed under the ultrapure nitrogen atmosphere. After the reaction, products were inserted into a dialysis membrane, Spectra/Por Membrane (Spectrum Laboratories, Inc., USA) with MWCO(molecular weight cut-off) 50000 and dialyzed by changing water every 3 hrs in the tertiary distilled water for a period of 24 hrs to remove unreacted polymers and solvents. The resulting dialyzed solution was frozen to −80° C. by using a deep freezer and then dried using a vacuum freeze dryer thereby removing the remaining solvents completely and obtaining powered product as a result. Thus obtained iron oxide power coated with polyvinylpyrolidone was well dissolved in water (powder B).

The picture of the powder B taken by Transmission Electron Microscope is shown in FIG. 1b and it was observed that uniform and spherical core of iron oxide nanoparticles of 10 nm in size were formed.

Dynamic light scattering analysis of the powder B in colloidal state revealed that the average size of iron oxide nanoparticles coated with polyvinylpyrolidone was 148.9 nm. The iron content of the powder B identified by elementary analysis was 5.7%

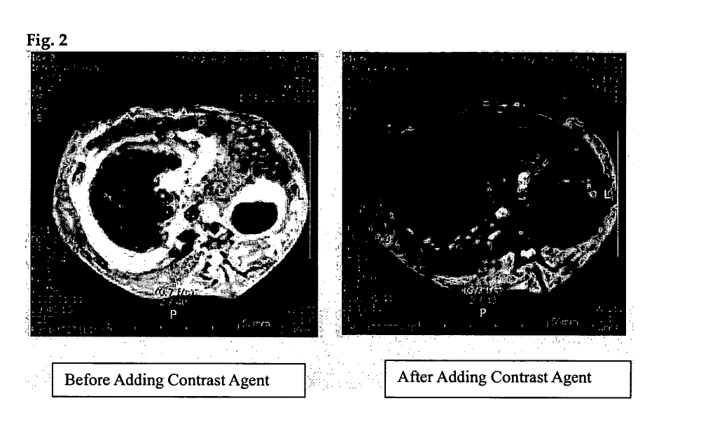

The above powder B in the amount of 0.24 g was dissolved in 1.4 mL of tertiary distilled water and was injected into a rabbit of 6 kg in body weight. The liver image contrast pictures taken via MRI before and after the administration of the powder B are shown in FIG. 2. The amount of administration per kg of body weight of the rabbit was in accordance with the commercially available Resovist composition (40 μmol Fe/kg).

### Comparative Example 1

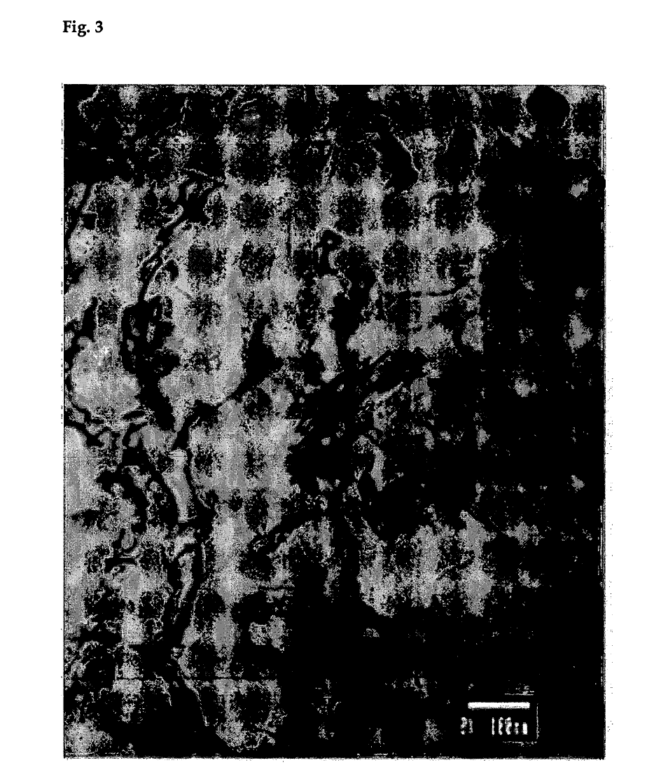

Experiment was performed the same as in example 1 except that K90 (MW; 3600000 g/mol, Sigma, USA) was used in place of polyvinylpyrolidone. As a result, there occurred aggregation and thus it was not possible to obtain uniform water soluble iron oxide nanoparticles (FIG. 3).

### Comparative Example 2

Experiment was performed the same as in example 1 except that 1 g of polyvinylpyrolidone and 0.143 mL of iron pentacarbonyl were used, wherein the molar ratio between polyvinylpyrolidone and iron pentacarbonyl was 1:11. As a result, there occurred aggregation and thus it was not possible to obtain uniform water soluble iron oxide nanoparticles.

### INDUSTIRAL APPLICABILITY

As stated above, the iron oxide nanoparticles of the present invention is coated with polyvinylpyrolidone, a biocompatible polymer, and thus it is water soluble and also its size can be controlled. Besides, it provides an excellent MRI image contrast effect thus can be used as MRI contrast agent. Further, it can be also used in targeted drug delivery, thermotherapy, magnetofection using DNA and magnetic nanoparticles coated with cationic molecules, therapeutic treatments of cell and tissues and the like.

The invention has been described in detail with reference to preferred embodiments thereof. However, it will be appreciated that those skilled in the art, upon consideration of the disclosure, may make modifications and improvements within the scope and spirit of the invention.

1. A water soluble iron oxide nanoparticle wherein its surface is coated with polyvinylpyrolidone.

2. In claim 1, the size of said nanoparticle is in the range of about 1 to 500 nm.

3. In claim 1, the molecular weight of said nanoparticle is in the range of about 3,000 to 100,000 g/mol.

4. In claim 1, said nanoparticle is used as contrast agent, targeted drug delivery, thermotherapy, magnetic nanoparticle, and therapeutic treatments of cells and tissues.

5. A method of manufacturing water soluble iron oxide nanoparticles comprising:
(a) adding iron oxide precursor at about 120 to 600° C. to a coating solution consisting of polyvinylpyrolidone and a polar organic solvent which dissolves the polyvinylpyrolidone;
(b) agitating the mixture at about 300 to 500 rpm for about 30 min to 72 hrs to obtain iron oxide nanoparticles with polyvinylpyrolidone coated on the surface.

6. In claim 5, the molecular weight of said polyvinylpyrolidone is in the range of about 3,000 to 100,000 g/mol.

7. In claim 5, said polar organic solvent is selected from the group consisting of carbitol, polyethyleneglycol, methoxy ethyleneglycol, dimethylformamide, propylenecarbonate, isopropylalcohol and glycerol.

8. In claim 5, said iron oxide precursor is selected from the group consisting of FeCup₃, Fe(acac)₃, Fe(CO)₅.

9. In claim 5, the molar ratio between said polyvinylpyrolidone and said iron oxide precursor is about 1:10 to 1:0.01.

10. In claim 5, the size of said nanoparticle is in the range of about 1 to 500 nm.

11. In claim 5, said manufacturing method is performed under nitrogen atmosphere.
